(19) United States
(12) Patent Application Publication
(10) Pub. No.: US 2004/0127719 A1
(43) Pub. Date: Jul. 1, 2004

(54) Alpha-isocyanocarboxylate solid support templates, method of preparation and for using the same

(76) Inventors: Kexin Yang, Louisville, KY (US); Boliang Lou, Louisville, KY (US)

Correspondence Address: John E. Vanderburgh, 608 Sunset Road, Louisville, KY, 40206, US

(21) Appl. No.: 10/094,599

(22) Filed: Mar. 8, 2002

(51) Int. Cl. C07F 7/02; C07F 7/10; G01N 33/53; G01N 33/543

(52) U.S. Cl. 548/406; 435/7.1; 436/518; 548/530; 556/415; 558/430

(57) ABSTRACT

A solid phase reaction template for the production of heterocyclic scaffolds in a reaction media, said solid phase component comprising an alpha-isocyanocarboxylate core compound linked to a solid substrate, the template having the formula:

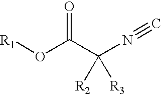

A method for the production of heterocyclic scaffolds using the template is disclosed

### FIELD OF INVENTION

The present invention relates to novel alpha-isocyanocarboxylate templates linked to insoluble materials and methods for producing novel heterocyclic classes of compounds through a plurality of chemical reactions utilizing alpha-isocyanocarboxylate templates on solid support.

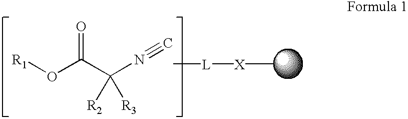

### BACKGROUND OF INVENTION

Isocyanide is a very unique calss of compounds. Its versatile utilizations in organic synthesis have been discussed in many research and review articals. In the past few years, we have witnessed that isocyanides have been used extensively as building blocks in combinatorial synthesis. They have been used in many reactions, especially in multiple components condensation reactions such as Ugi reaction, to generate many novel scaffolds. However, only a dozen or so isocyanides are commercially available and the extreme unpleasant odor of isocyanides makes them not easy to handle. Therefore, there is a need to develop general methodologies for deriving structurally diversified isocyanides from readily available starting materials such as primary amines or other amino group containing molecules. Furthermore, making the isocyanides polymer-bound will eliminate the handling hassle and certainly will find widespread applications in solid phase combinatorial synthesis.

### SUMMARY OF INVENTION

The present invention relates to alpha-isocyanocarboxylate core compounds of the general formula as follows:

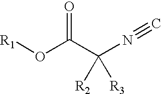

The core compounds are linked to appropriate insoluble substrates to create solid support templates of Formula 1, wherein

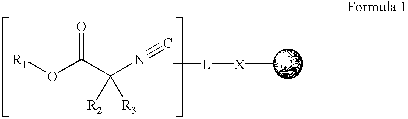

The solid support, represented by the shaded circle, is intended to include the following:

a.) beads, pellets, disks, fibers, gels, or particles such as cellulose beads, pre-glass beads, silica gels, polypropylene beads, polyacrylamide beads, polystyrene beads that are lightly cross-linked with 1-2% divinylbenzene and optionally grafted with polyethylene glycol and optionally functionalized with amino, hydroxy, carboxy or halo groups; and

b.) soluble supports such as low molecular weight non-cross-linked polystyrene and polyethylene glycol.

The term solid support is used interchangeably with the term resin or bead in this invention and is intended to mean the same thing.

X is an atom or a functional group connecting the polymer and the linker L, having a structure such as but not limited to oxygen, ester, amide, sulfur, silicon and carbon;

L is a suitable linker, a multifunctional chemical monomer in which one functional group reacts with the polymer to form a covalent bond (X) and the other functional groups react with one of R groups (R₁, R₂, R₃) through a plurality of chemical reactions to provide the desired templates for further chemistry. Both X and R groups can be represented within a suitable monomer L, such as an amino acid; Commercially available resins, like Wang and Hydroxymethyl polystyrene, are useful in this method. The linkers present in these resins allow the cleavage of final products by a variety of mild chemical conditions that allow isolation of compounds of this invention. The hydroxymethyl polystyrene resin and the Wang resin are examples of solid phase supports used in the preparation of compounds of this invention. Other known or commercially available solid phase supports work in this method and are considered to lie within the scope of this invention.

R₁ is selected from a group consisting of a covalent bond attached to the L, hydrogen, substituted alkylsilyl, substituted alkyl, substituted alkenyl;

R₂ and R₃ are independently selected from a group consisting of a covalent bond attached to the L, hydrogen, substituted alkyl, substituted alkenyl, substituted alkenylaryl, substituted alkynyl, substituted aryl, substituted alkylaryl, substituted arylalkyl, substituted heteroaryl, substituted heteroarylalkyl, substituted saturated heterocyclyl, substituted cycloalkyl and substituted alkylcycloalkyl; or

R₁ and R₂, or R₁ and R₃, or R₂ and R₃ taken in combination, are substituted cycloalkyl and substituted saturated heterocyclyl;

### DETAIL DESCRIPTION OF THE INVENTION

As used above, and through the description of the invention, the following terms, unless otherwise indicated, shall be understood to have the following meanings:

Definitions

“Alkyl” means a saturated aliphatic hydrocarbon group which may be straight or branched and having about 1 to about 20 carbons in the chain. Branched means that a lower alkyl group such as methyl, ethyl, or propyl is attached to a linear alkyl chain. Preferred straight or branched alkyl groups are the “lower alky” groups which are those alkyl groups having from 1 to about 6 carbon atoms.

“Alkenyl” means an aliphatic hydrocarbon group defined the same as for “alkyl” plus at least one double bond between two carbon atoms anywhere in the hydrocarbon.

“Alkynyl” means an aliphatic hydrocarbon group defined the same as for “alkyl” plus at least one triple bond between two carbon atoms anywhere in the hydrocarbon.

“Aryl” represents an unsubstituted, mono-, di- or trisubstituted monocyclic, polycyclic, biaryl aromatic groups covalently attached at any ring position capable of forming a stable covalent bond, certain preferred points of attachment being apparent to those skilled in the art. Aryl thus contains at least one ring having at least 5 atoms, with up to two such rings being present, containing up to 10 atoms therein, with alternating (resonating) double bonds between adjacent carbon atoms. Aryl groups may likewise be substituted with 0-3 groups selected from Rₛ. The definition of aryl includes but is not limited to phenyl, biphenyl, indenyl, fluorenyl, naphthyl (1-naphtyl, 2-naphthyl).

Heteroaryl is a group containing from 5 to 10 atoms, 1-4 of which are heteroatoms, 0-4 of which heteroatoms are nitrogen, and 0-1 of which are oxygen or sulfur, said heteroaryl groups being substituted with 0-3 groups selected from Rₛ, The definition of heteroaryl includes but is not limited to pyridyl, furyl, thiophenyl, indolyl, thiazolyl, imidazolyl, benzimidazolyl, tetrazolyl, pyrazinyl, pyrimidyl, quinolyl, isoquinolyl, benzofuryl, isothiazolyl, benzothienyl, pyrazolyl, isoindolyl, isoindolyl, purinyl, carbazolyl, oxazolyl, benzthiazolyl, benzoxazolyl, quinoxalinyl, quinazolinyl, and indazolyl.

“Cycloalkyl” means a saturated carbocyclic group having one or more rings and having 3 to about 10 carbon atoms. Preferrd cycloalkyl groups include cyclopropyl, cyclobutyl, cyclopentyl, cyclohexyl, cycloheptyl, and decahydronaphthyl.

“heterocyclyl” means an about 4 to about 10 member monocyclic or multicyclic ring system wherein one or more of the atoms in the ring system is an element other than carbon chosen amongst nitrogen, oxygen or sulfur. The heterocyclyl may be optionally substituted by one or more alkyl group substituents. Examplary heterocyclyl moieties include quinuclidine, pentamethylenesulfide, tetrahedropyranyl, tetrahydrothiophenyl, pyrrolidinyl or tetrahydrofuranyl.

“Saturated” means that the atom possesses the maximum number of single bonds either to hydrogen or to other atoms, eg. a carbon atom is sp³ hybridized.

“Unsaturated” means that the atom possesses less than the maximum number of single bonds either to hydrogen or to other atoms, eg. a carbon atom is sp² or sp³ hybridized.

“Substituted” means the attachment of any of the following groups, including:

(i) H

(ii) alkyl

(iii) aryl

(iv) amino, amidino, bromo, chloro, carboxy, carboxamido, thiocarboxy, cyano, fluoro, guanidino, hydroxy, iodo, nitro, oxo, thiol, trihalomethyl, trihalomethoxy

(v) N-(C₁-C₆alkyl)amidino and N-aryl amidino

(vi) N-(C₁-C6alkyl)guanidino and N-aryl guanidino

(vii) C₁-C₆alkylamino and arylamino

(viii) N,N′-(C₁-C₆dialkyl)amino, N,N′-diarylamino and N-(C₁-C₆alkyl)-N′-(aryl)-amino

(ix) C₁-C₆alkylarylamino and arylC₁-C₆alkylamino

(x) 4-, 5-, 6-, or 7-membered azacycloalkanes

(xi) C₁-C₆alkyloxy and aryloxy

(xii) C₁-C₆alkylaryloxy and arylC₁-C₆alkyloxy

(xiii) C₁-C₆alkylarylthio and arylC₁-C₆alkylthio

(Xiv) C₁-C₆alkylcarbonyl and arylcarbonyl

(xv) C₁-C₆alkylarylcarbonyl and arylC₁-C₆alkylcarbonyl

(xvi) C₁-C6alkoxycarbonyl and aryloxycarbonyl

(XVii) C₁-C₆alkylaryloxycarbonyl and arylC₁-C₆alkyloxycarbonyl

(XViii) C₁-C₆alkylarylthiocarbonyl and arylC₁-C₆alkylthiocarbonyl

(xix) N-mono-(C₁-C₆alkyl) and N,N′-di-(C₁-C₆alkyl)aminocarbonyl

(xx) N-mono-(aryl) and N,N′-di-(aryl)aminocarbonyl

(xxi) N,N′-(C₁-C₆alkyl)(aryl)aminocarbonyl

(xxii) N-mono-(C₁-C₆alkylaryl) and N,N′-di-(arylC₁-C₆alkyl)aminocarbonyl

(xxiii) N,N′-(C₁-C₆alkyl)(arylC₁-C₆alkyl)aminocarbonyl

(xxiv) N,N′-(aryl)(arylC₁-C₆alkyl)aminocarbonyl

(xxv) C₁-C₆alkylcarbonylamino and arylcarbonylamino

(xxvi) C₁-C₆alkylarylcarbonylamino and arylC₁-C₆alkylcarbonylamino

(xxvii) C₁-C₆alkoxycarbonylamino and aryloxycarbonylamino

(xxviii) C₁-C₆alkylaryloxycarbonylamino and arylC₁-C₆alkyloxycarbonylamino

(xxix) C₁-C₆alkylarylthiocarbonylamino and arylC₁-C₆alkylthiocarbonylamino

(xxx) N-mono-(C₁-C₆alkyl) and N,N′-di-(C₁-C₆alkyl)aminocarbonylamino

(xxxi) N-mono-(aryl) and N,N′-di-(aryl)aminocarbonylamino

(xxxii) N,N′-(C₁-C₆alkyl)(aryl)aminocarbonylamino

(xxxiii) N-mono-(C₁-C₆alkylaryl) and N,N′-di-(arylC₁-C6alkyl)aminocarbonylamino

(xxxiv) N,N′-(C₁-C₆alkyl)(arylC₁-C₆alkyl)aminocarbonylamino

(xxxv) N,N′-(aryl)(arylC₁-C₆alkyl)aminocarbonylamino

(xxxvi) C₁-C₆alkylcarbonyloxy and arylcarbonyloxy

(xxxvii) C₁-C₆alkylarylcarbonyloxy and arylC₁-C₆alkylcarbonyloxy

(xxxviii) C₁-C₆alkoxycarbonyloxy and aryloxycarbonyloxy

(xxxix) C₁-C₆alkylaryloxycarbonyloxy and arylC₁-C₆alkyloxycarbonyloxy

(xl) C₁-C₆alkylarylthiocarbonyloxy and arylC₁-C₆alkylthiocarbonyloxy

(xli) N-mono-(C₁-C₆alkyl) and N,N′-di-(C₁-C₆alkyl)aminocarbonyloxy

(xlii) N-mono-(aryl) and N,N′-di-(aryl)aminocarbonyloxy

(xliii) N,N′-(C₁-C₆alkyl)(aryl)aminocarbonyloxy

(xliv) N-mono-(C₁-C₆alkylaryl) and N,N′-di-(arylC₁-C6alkyl)aminocarbonyloxy

(xlv) N,N′-(C₁-C₆alkyl)(arylC₁-C₆alkyl) aminocarbonyloxy and N,N′-(aryl)(arylC₁-C₆alkyl)aminocarbonyloxy

(xlvi) C₁-C₆alkylsulfoxy and arylsulfoxy

(xlvii) C₁-C₆alkylarylsulfoxy and arylC₁-C₆alkylsulfoxy

(xlviii) C₁-C₆alkylsulfonyl and aryl sulfonyl

(xlix) C₁-C₆alkylarylsulfonyl and arylC₁-C₆alkylsulfonyl

(l) C₁-C₆alkylsulfonamido and arylsulfonamido

(li) C₁-C₆alkylarylsulfonamido and arylC₁-C₆alkylsulfonamido

(lii) C₁-C₆alkylaminosulfonyl and arylaminosulfonyl

(liii) C₁-C₆alkylarylaminosulfonyl and arylC₁-C₆alkylaminosulfonyl

(liv) C₁-C₆alkylaminosulfonamido and arylaminosulfonamido

(lv) C₁-C₆alkylarylsulfonamido and arylC₁-C₆alkylsulfonamido

“Alkyl” and “aryl” used for any of the groups in the above list also means substituted alkyl or substituted aryl, where substituted means groups selected from the same list. Alkyl groups also include alkenyl and alkynyl groups in the above list of substituents.

Preferred Embodiments

A preferred solid support template of the present invention is the template of Formula 1 wherein R₁ is a covalent bond and the linker is directly attached to the carboxylate, can be presented as Formula 1a, wherein

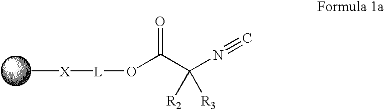

R₂ and R₃ are independently selected from a group consisting of a covalent bond attached to the L, hydrogen, substituted alkyl, substituted alkenyl, substituted alkenylaryl, substituted alkynyl, substituted aryl, substituted alkylaryl, substituted arylalkyl, substituted heteroaryl, substituted heteroarylalkyl, substituted saturated heterocyclyl, substituted cycloalkyl and substituted alkylcycloalkyl; or

R₂ and R₃ taken in combination, are substituted cycloalkyl and substituted saturated heterocyclyl.

Another preferred solid support template of the present invention is the template of Formula 1b wherein R3 is hydrogen, the linker L is attached to R₂, can be presented as Formula 1b, wherein

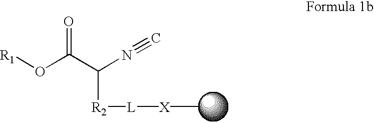

R₁ is hydrogen, substituted alkylsilyl, substituted alkyl, substituted alkenyl.

R₂ is a covalent bond, or a multifunctional chemical monomer possessing at least two attachment points linking to alpha-carbon and the L, selected from a group consisting substituted alkyl, substituted alkenyl, substituted alkenylaryl, substituted alkynyl, substituted aryl, substituted alkylaryl, substituted heteroaryl, substituted alkylheteroaryl, substituted saturated heterocyclyl, substituted cycloalkyl and substituted alkylcycloalkyl; or

R₁ and R₂ taken in combination, is substituted saturated heterocyclyl.

Solid Support

Solid support is a substrate consisting of a polymer, cross-linked polymer, functionalized polymeric pin, or other insoluble material. These polymers or insoluble materials have been described in literature and are known to those who are skilled in the art of solid phase synthesis (Stewart J M, Young J. D.; Solid Phase Peptide Synthesis, 2nd Ed; Pierce Chemical Company: Rockford. Ill., 1984). Some of them are based on polymeric organic substrates such as polyethylene, polystyrene, polypropylene, polyethylene glycol, polyacrylamide, and cellulose. Additional types of supports include composite structures such as grafted copolymers and polymeric substrates such as polyacrylamide supported within an inorganic matrix such as kieselguhr particles, silica gel, and controlled pore glass.

Examples of suitable support resins and linkers are given in various reviews (Barany, G.; Merrifield, R. B. “Solid Phase Peptide Synthesis”, in “The Peptides—Analysis, Synthesis, Biology”. Vol 2, [Gross, E. and Meienhofer, J., Eds.], Academic Press, Inc., New York, 1979, pp 1-284; Backes, B. J.; Ellman, J. A. Curr. Opin. Chem. Biol. 1997. 1, 86; James, I. W., Tetrahedron 1999, 55, 4855-4946) and in commercial catalogs (Advanced ChemTech, Louisville, Ky.; Novabiochem, San Diego, Calif.). Some examples of particularly useful functionalized resin/linker combinations that are meant to be illustrative and not limiting in scope are shown below:

(1) Aminomethyl polystyrene resin (Mitchell, A. R., et al., J. Org. Chem., 1978, 43, 2845):

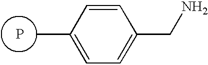

This resin is the core of a wide variety of synthesis resins. The amide linkage can be formed through the coupling of a carboxylic acid to amino group on solid support resin under standard peptide coupling conditions. The amide bond is usually stable under the cleavage conditions for most acid labile, photo labile and base labile or nucleophilic linkers.

(2) Wang resin (Wang, S. S.; J. Am. Chem. Soc. 1973, 95, 1328

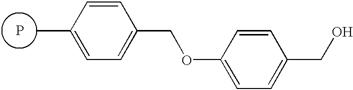

-1333). Wang resin is perhaps the most widely used of all resins for acid substrates bound to the solid support resin. The linkage between the substrate and the polystyrene core is through a 4-hydroxybenzyl alcohol moiety. The linker is bound to the resin through a phenyl ether linkage and the carboxylic acid substrate is usually bound to the linker through a benzyl ester linkage. The ester linkage has good stability to a variety of reaction conditions, but can be readily cleaved under acidic conditions, such as by using 25% TFA in DCM.

(3) Rink resin (Rink, H.; Tetrahedron Lett. 1987, 28, 3787).

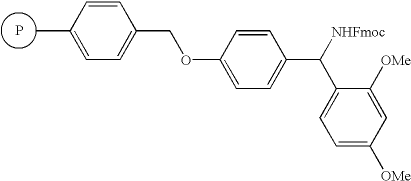

Rink resin is used to prepare amides utilizing the Fmoc strategy. It has also found tremendous utility for a wide range of solid phase organic synthesis protocols. The substrate is assembled under basic or neutral conditions, then the product is cleaved under acidic conditions, such as 10% TFA in DCM.

(4) Knorr resin (Bernatowicz, M. S., et al. Tetrahedron Lett., 1989, 30, 4645).

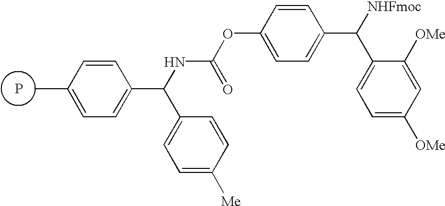

Knorr resin is very similar to Rink resin, except that the linker has been modified to be more stable to TFA.

(5) PAL resin (Bernatowicz, M. S., et al. Tetrahedron lett., 1989, 30, 4645).

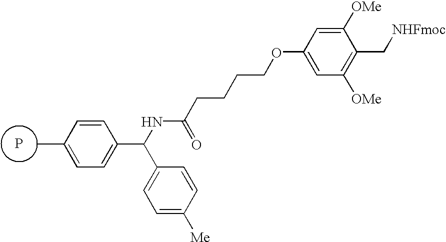

(6) HMBA-MBHA Resin (Sheppard, R. C., et al., Int. J. Peptide Protein Res. 1982, 20, 451).

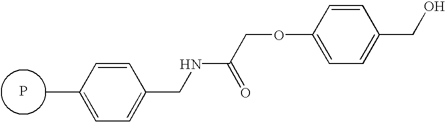

(7) HMPA resin. This also is an acid labile resin which provides an alternative to Wang resin and represented as:

(8) Benzhydrylamine copoly(styrene-1 or 2%-divinylbenzene) which referred to as the BHA resin (Pietta, P. G., et al., J. Org. Chem. 1974, 39, 44).

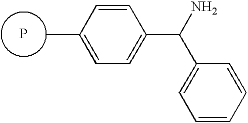

(9) Methyl benzhydrylamine copoly(styrene-1 or 2%-divinylbenzene) which is referred to as MBHA and represented as:

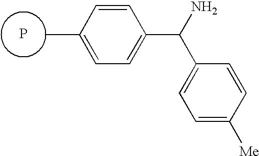

(10) Trityl and functionalized Trityl resins, such as aminotrityl resin and amino-2-chlorotrityl resin (Barlos, K.; Gatos, D.; Papapholiu, G.; Schafer, W.; Wenqing, Y.; Tetrahedron Lett. 1989, 30, 3947).

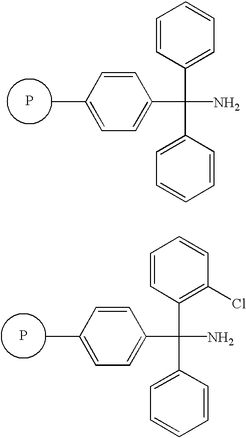

(11) Sieber amide resin (Sieber, P.; Tetrahedron Lett. 1987, 28, 2107).

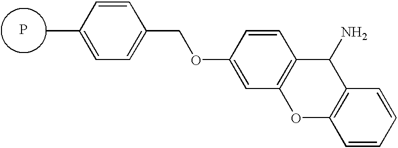

(12) Rink acid resin (Rink, H., Tetrahedron Lett., 1987, 28, 3787).

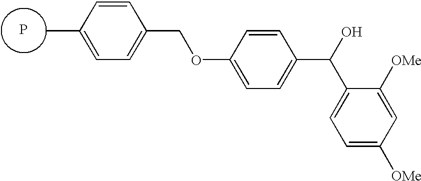

(13) HMPB-BHA resin (4-hydroxymethyl-3-methoxyphenoxybutyric acid-BHA Florsheimer, A.; Riniker, B. in “Peptides 1990; Proceedings of the 21^{st} European Peptide Symposium”, [Giralt, E. and Andreu, D. Eds.], ESCOM, Leiden, 1991, pp 131.

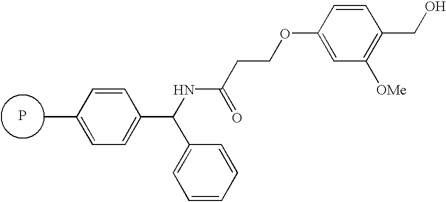

(14) Merrifield resin—Chloromethyl co-poly(styrene-1 or 2%-divinylbenzene) which can be represented as:

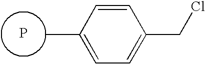

A carboxylic acid substrate is attached to the resin through nucleophilic replacement of chloride under basic conditions. The resin is usually stable under acidic conditions, but the products can be cleaved under basic and nucleophilic conditions in the presence of amine, alcohol, thiol and H₂O.

(15) Hydroxymethyl polystyrene resin (Wang, S. S., J. Org. Chem., 1975, 40, 1235).

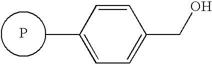

The resin is an alternative to the corresponding Merrifield resin, whereas the substrate is attached to a halomethylated resin through nucleophilic displacement of halogen on the resin, the attachment to hydroxymethylated resins is achieved by coupling of activated carboxylic acids to the hydroxy group on the resin or through Mitsunobu reactions. The products can be cleaved from the resin using a variety of nucleophiles, such as hydroxides, amines or alkoxides to give carboxylic acids, amides and esters.

(16) Oxime resin (DeGrado, W. F.; Kaiser, E. T.; J.Org. Chem. 1982, 47,

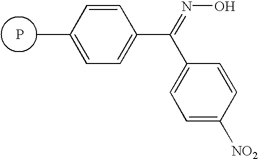

3258).

This resin is compatible to Boc chemistry. The product can be cleaved under basic conditions.

(16) Photolabile resins (e.g. Abraham, N. A. et al.; Tetrahedron Lett. 1991, 32, 577). The products can be cleaved from these resins photolytically under neutral or mild conditions, making these resins useful for preparing pH sensitive compounds. Examples of the photolabile resins include:

(a) ANP resin:

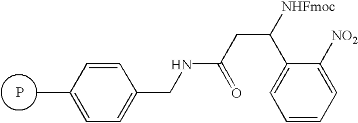

(b) alpha-bromo-alpha-methylphenacyl polystyrene resin:

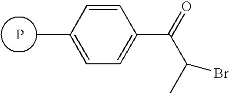

(17) Safety catch resins (see resin reviews above; Backes, B. J.; Virgilio, A. A.; Ellman, J. Am. Chem. Soc. 1996, 118, 3055-6). These resins are usually used in solid phase organic synthesis to prepare carboxylic acids and amides, which contain sulfonamide linkers stable to basic and nucleophilic reagents. Treating the resin with haloacetonitriles, diazomethane, or TMSCHN₂ activates the linkers to attack, releasing the attached carboxylic acid as a free acid, an amide or an ester depending on whether the nucleophile is a hydroxide, amine, or alcohol, resepectively. Examples of the safty catch resins include:

(a) 4-sulfamylbenzoyl-4′-methylbenzhydrylamine resin:

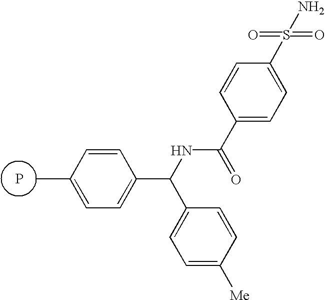

(b) 4-sulfamylbutryl-4′-methylbenzhydrylamine resin:

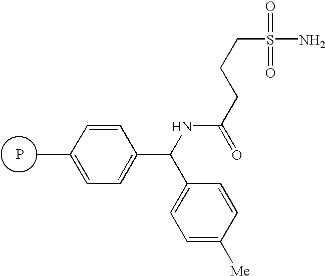

(18) TentaGel resins:

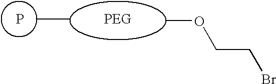

TentaGel resins are polyoxyethyleneglycol (PEG) grafted (Tentagel) resins (Rapp, W.; Zhang, L.; Habich, R.; Bayer, E. in “Peptides 1988; Proc. 20^{tth} European Peptide Symposium” [Jung,G. and Bayer, E., Eds.], Walter de Gruyter, Berlin, 1989, pp 199-201. TentaGel resins, e.g. TentaGel S Br resin can swell in a wide variety of solvents and the bead size distribution is very narrow, making these resins ideal for solid phase organic synthesis of combinatorial libraies. TentaGel S Br resin can immobilize carboxylic acids by displacing the bromine with a carboxylic acid salt. The products can be released by saponification with dilute aqueous base.

(19) Resins with silicon linkage (Chenera, B.; Finkelstein, J. A.; Veber, D. F.; J. Am. Chem. Soc. 1995, 117, 11999-12000; Woolard, F. X.; Paetsch, J.; Ellman, J. A.; J. Org. Chem. 1997, 62, 6102-3). Some examples of these resins contain protiodetachable arylsilane linker and traceless silyl linker. The products can be released in the presence of fluoride.

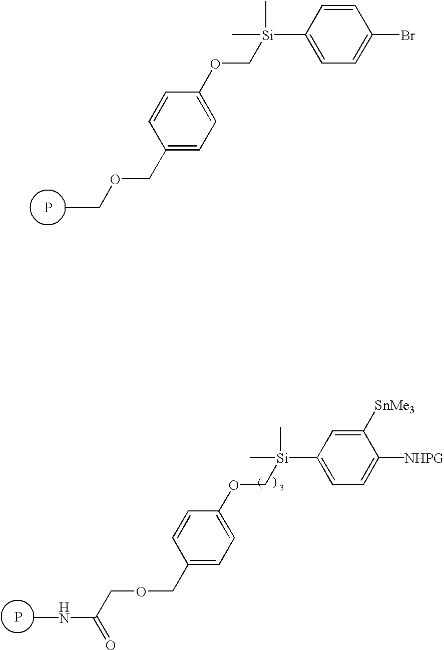

Also useful as a solid phase support in the present invention are solubilizable resins that can be rendered insoluble during the synthesis process as solid phase supports. Although this technique is frequently referred to as “Liquid Phase Synthesis”, the critical aspect for our process is the isolation of individual molecules from each other on the resin and the ability to wash away excess reagents following a reaction sequence. This also is achieved by attachment to resins that can be solubilized under certain solvent and reaction conditions and rendered insoluble for isolation of reaction products from reagents. This latter approach, (Vandersteen, A. M.; Han, H.; Janda, K. D.; Molecular Diversity, 1996, 2, 89-96.) uses high molecular weight polyethyleneglycol as a solubilizable polymeric support and such resins are also used in the present invention.

Experimental Details

The following sections described details of experiments related to the preparation of polymer-bound α-isocyanocarboxylate templates and their applications in syntheses of novel heterocyclic scaffolds. The examples are by way of illustration of various aspects of the present invention and are not intended to be limiting thereof.

Reagents and Test Methods

Solvents and chemicals were purchased from commercial sources such as Advanced ChemTech, Aldrich, Fisher Scientific, Lancaster, etc. and used without further purification unless otherwise indicated. Resins with typical loading level ranging from 0.30 to 1.0 mmol/g were purchased from Advanced ChemTech and used directly. IR spectra were obtained on a Midac M1700 and absorbencies are listed in inverse centimeters. LC/MS analyses were performed on a Hewlett-Packard 1100 HPLC/Micromass Platform II electronspray mass spectrometer system. A photodiode array detector and an evaporative light scattering detector (Sedex 55) were also incorporated with the LC/MS system for more accurate evaluation of sample purity. Reverse phase columns were purchased from YMC, Inc. (ODS-A, 3 μm, 120 Å, 4.0×50 mm). Two mobile phase solvents were used for LC/ MS analysis. Solvent A consisted of 97.5% acetonitrile, 2.5% H₂O, and 0.05% TFA. Solvent B consisted of 97.5% H₂O, 2.5% acetonitrile, and 0.05% TFA. Analytical data were typically acquired at a mobile phase flow rate of 1.8 ml/min involving a 2 minute gradient from solvent B to solvent A with 5 minute run times. H¹-NMR analysis was carried out on a Varian 300 MHz NMR spectrometer.

Preparation of the Templates

The synthesis of the isocyanide template Formula 1a, wherein R₁ is a covalent bond and the linker is directly attached to the carboxylate, was illustrated in the following Scheme 1.

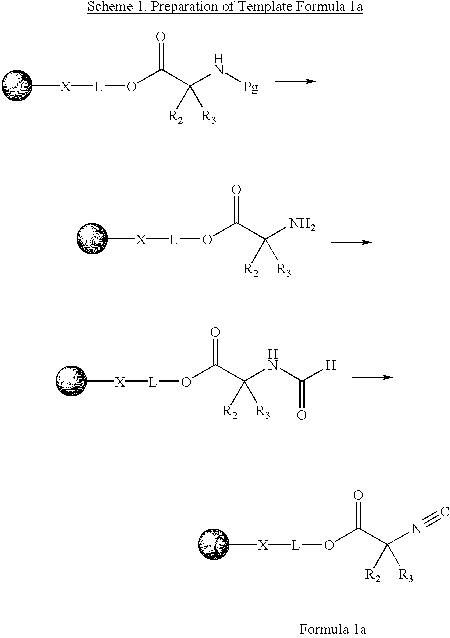

Scheme 1. Preparation of Template Formula 1a

Depending on the nature of the polymer linker, the appropriate protecting group (Pg) can be removed using standard protocol. For example, Fmoc group is removed by treatment with 20% piperdine in DMF at room temperature for 30 minutes while Boc group is removed by treatment with 20% trifluoroacetic acid in DCM at room temperature for 30 minutes. After removing the protecting, the amino group was formylated by treating the resin with formic acid and diisopropylcarbodiimide. Then, the formylated α-amino group was transformed into the desired isocyano group by treatment with triphenylphosphine, carbontetrachloride, and triethylamine. We also found that the formation of isocyano group from formylated amino group can also be achieved by treatment of triphenylphosphine, trichloroacetonitrile, and triethylamine.

Based on the method described above, a variety of polymer-bound isocyanocarboxylate were prepared. Some examples are shown in FIG. 1.

[image]

Representative procedures for preparation of resins 1 and 11 are given as example 1 and 2.

### EXAMPLE 1

### Preparation of 2-Isocyano-3-phenyl propionate Wang Resin (1)

Fmoc-Phe-Wang resin (10 g, 0.7 mmol/g) was treated with 20% piperidine solution in DMF (100 mL) at room temperature for 45 minutes. The resin was then collected by filtration and washed successively with DMF, DCM, and MeOH several times, then dried at room temperature under vacuum. Sample of the above resin was analyzed by infrared (IR) spectroscopy. IR (KBr): 1732 cm⁻¹ (C═O); 3300-3500 cm⁻¹ (NH₂).

The de-protected resin from above was suspended in DCM (100 mL), followed by addition of 10 molar equivalent of formic acid (3.2 g, ˜70 mmol). Then, 10 molar equivalent of DIC (14 g, ˜70 mmol) was added dropwise to the stirring suspension (Caution: the reaction was exothermic! DCM will start to gently refluxing during the addition of DIC.). The suspension was stirred for an additional hour after the addition of DIC. The resin was then collected, washed, and dried as usual. IR (KBr): 1741 cm⁻¹ (C═O of ester linkage); 1693 cm⁻¹ (C═O of newly formed amide).

The formylated resin was suspended in DCM (100 mL), followed by addition of 5 molar equivalent each of carbon tetrachloride (5.4 g, ˜35 mmol) and triphenylphosphine (9.2 g, ˜35 mmol). The mixture was stirred at room temperature under a nitrogen atmosphere for about 2 hours. The mixture turned gradually from colorless to light yellow. 5 molar equivalent of TEA (4.5 g, ˜35 mmol) was then added to the mixture. The reaction mixture turned dark rapidly. 10 minutes later, the resin was collected, washed and dried as usual. IR (KBr): 1750 cm⁻¹ (C═O of ester linkage); 2145 cm⁻¹ (N≡C).

### EXAMPLE 2

### Preparation of 2-Isocyano-4-methyl pentanoate Merrifield Resin (11)

Boc-Leu-Merrifield resin was treated with 20% trifluoroacetic acid solution in DCM (about 10 mL per gram of resin) at room temperature for 45 minutes. The resin was collected by filtration and washed with DCM, 10% TEA/DCM and MeOH several times, then dried at room temperature under vacuum. IR spectrum of the resin showed C═O and NH₂ stretches at 1734 cm⁻¹ and 3300-3500 cm⁻¹ respectively.

The de-protected resin from above was suspended in DCM (about 10 mL per gram of resin), followed by addition of 10 molar equivalent of formic acid. Then, 10 molar equivalent of DIC was added to the stirring suspension in small portions (Caution: the reaction was exothermic! DCM will start to gently refluxing during the addition of DIC.). The suspension was stirred for an additional hour after the addition of DIC. The resin was then collected, washed, and dried as usual. Its IR spectrum showed a new amide C═O stretch at 1681 cm⁻¹.

The formylated resin was suspended in DCM (about 10 mL per gram of resin), followed by addition of 5 molar equivalent each of carbon tetrachloride and triphenylphosphine. The mixture was stirred at room temperature under a nitrogen atmosphere for about 2 hours. The mixture turned gradually from colorless to light yellow. 5 molar equivalent of TEA was added to the mixture. It turned brown rapidly. 10 minutes later, the resin was collected, washed and dried as usual. The IR showed disappearance of the amide C═O absorption and appearance of the N≡C absorption at 2145 cm⁻¹.

Similarly, the methodology described above for preparation of template Formula 1a can also be applied to prepare template of Formula 1b, as shown in Scheme 2.

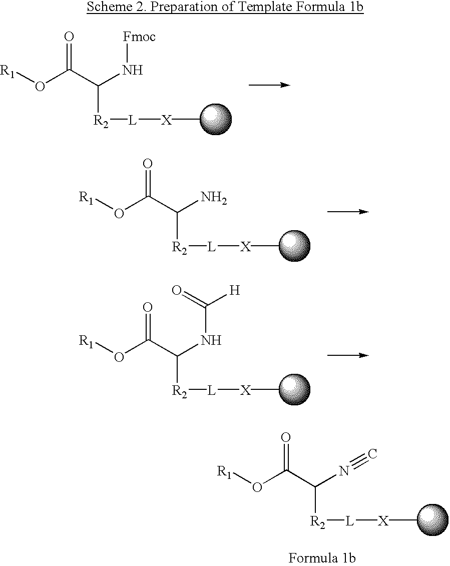

As an example of Formula 1b, resin-bound glutamic acid derivative (13) was converted to its corresponding isocyanide (14) according to procedures described for resin (1), as shown in the following Scheme 3.

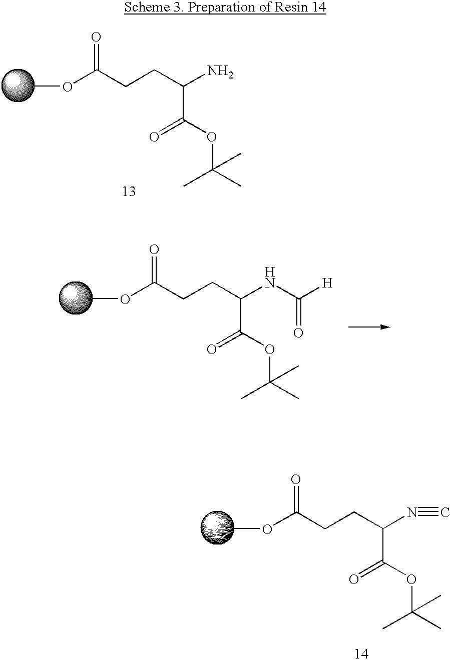

### EXAMPLE 3

### Preparation of 4-Isocyano-4-(t-butoxycarbonyl)butanoate Wang Resin (14)

N-Fmoc-4-amino-4-(t-butoxycarbonyl)-butyrate-Wang resin (5 g, 0.7 mmol/g) was treated with 20% piperidine solution in DMF (50 mL) at room temperature for 45 minutes. The resin was then collected by filtration and washed successively with DMF, DCM, and MeOH several times, then dried at room temperature under vacuum. Sample of the above resin was analyzed by infrared (IR) spectroscopy. IR (KBr): 1734 cm⁻¹ (C═O); 3300-3500 cm⁻¹ (NH₂).

The de-protected resin from above was suspended in DCM (50 mL), followed by addition of 10 molar equivalent of formic acid (1.6 g, ˜35 mmol). Then, 10 molar equivalent of DIC (7.0 g, ˜35 mmol) was added dropwise to the stirring suspension (Caution: the reaction was exothermic! DCM will start to gently refluxing during the addition of DIC.). The suspension was stirred for an additional hour after the addition of DIC. The resin was then collected, washed, and dried as usual. IR (KBr): 1734 cm⁻¹ (C═O of ester linkage); 1696 cm⁻¹ (C═O of newly formed amide).

The formylated resin was suspended in DCM (50 mL), followed by addition of 5 molar equivalent each of carbon tetrachloride (2.7 g, ˜17.5 mmol) and triphenylphosphine (4.6 g, ˜17.5 mmol). The mixture was stirred at room temperature under a nitrogen atmosphere for about 2 hours. The mixture turned gradually from colorless to light yellow. 5 molar equivalent of TEA (2.3 g, ˜17.5 mmol) was then added to the mixture. The reaction mixture turned dark rapidly. 10 minutes later, the resin was collected, washed and dried as usual. IR (KBr): 1728 cm⁻¹ (C═O of ester linkage); 2146 cm⁻¹ (N≡C).

Synthesis of Novel Heterocycle Scaffolds Using the Templates

In parallel with the rich chemistry of isocyanids in conventional solution phase reactions, polymer-bound α-isocyanocarboxylates demonstrated their versatile utilities in solid phase syntheses, especially in solid phase multi-component condensation reactions to afford many interesting heterocyclic scaffolds. Some example reaction schemes are shown in the following sections.

1. Synthesis of 2-imidazolines:

Polymer-bound isocyanides have been used in the Ugi four-component condensation reaction to give α-acylaminoamides, which can be then transferred into a variety of interesting heterocyclic structures, such as imidazole, gama-lactam, hydantoin, 1,4-benzodiazepine, diketopiperazine. However, the use of resin-bound α-isocyanocarboxylates in exploiting combinatorial synthesis was reported only recently.^{1,2} We examined our resin-bound α-isocyanocarboxylates under the standard Ugi reaction conditions. For example, 3-phenyl-2-isocyanopropionate Wang resin was treated with p-anisaldehyde (10 equiv.) and isobutylamine (10 equiv.) in the presence of acetic acid (10 equiv.) in 1:1 THF/MeOH at rt for 48 h. We surprisingly found that 1,4,4,5-tetrasubstituted 2-imidazoline derivative, (15) as shown in Scheme 4, was obtained as a major product upon cleavage with 20% TFA/DCM with no observation of the expected normal Ugi product, α-acylaminoamides (16).

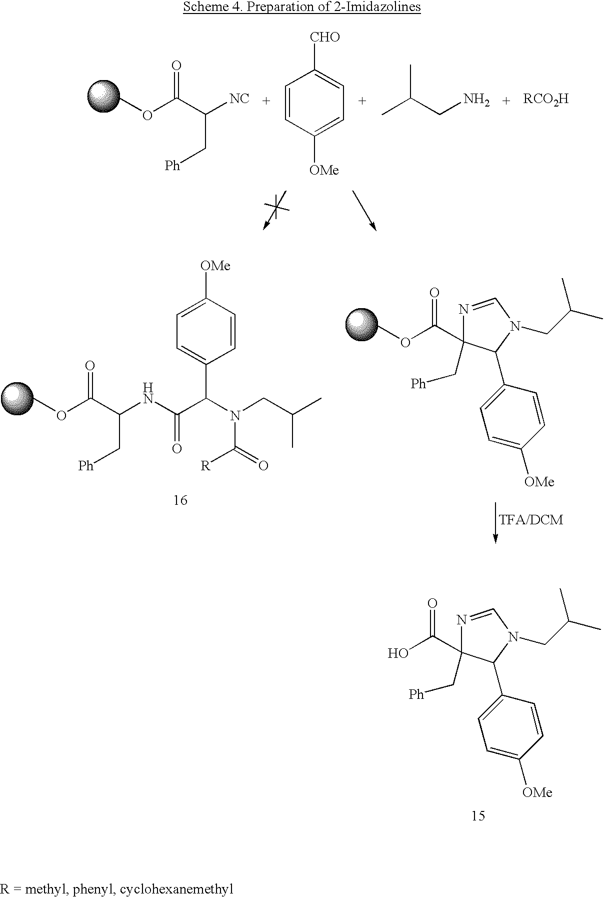

Further studies on this reaction in the presence of other carboxylic acids, such as phenylacetic acid or benzoic acid, proved that the acid input served only as a promoter for imine formation. This observation prompted us to investigate a variety of Lewis acids for use in the reactions. We found that the reactions proceeded smoothly in the presence of ZnCl₂, and it was compatible with a wide range of aldehydes and amines. By using this protocol as shown in Scheme 5, a 2-imidazoline library has been synthesized in satisfactory yields and purity. Table 1 illustrates some representative members selected from the library.

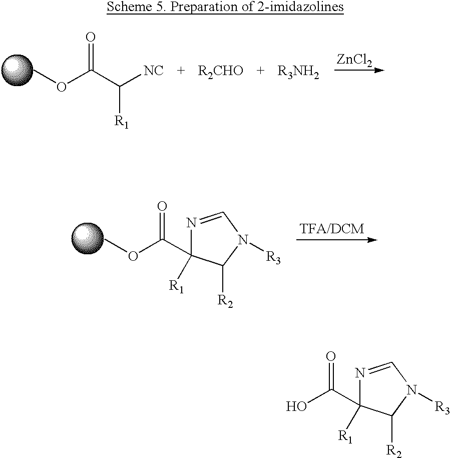

TABLE 1
2-Imidazolines From 3-component condensation reactions

| 2-Imidazolines | Yield(%) | Purity(%) |
|---|---|---|
| 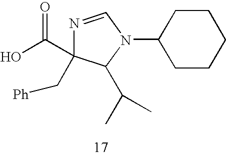 | 84 | 90 |
| 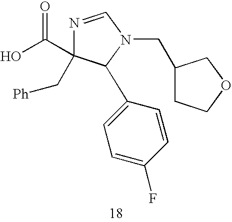 | 57 | 80 |
| 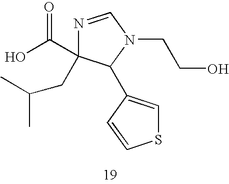 | 70 | 90 |
| 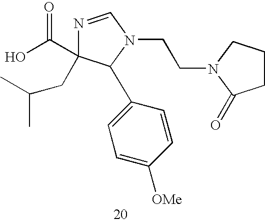 | 62 | 60 |
| 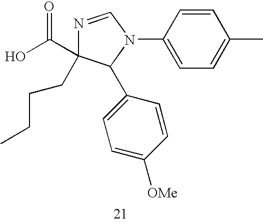 | 70 | 90 |
| 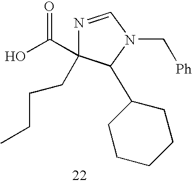 | 75 | 75 |
| 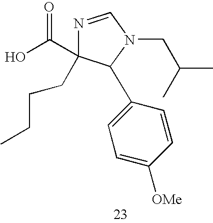 | 92 | 90 |
| 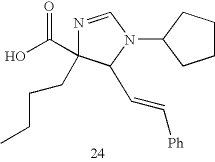 | 90 | 90 |
| 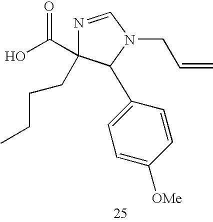 | 100 | 90 |
| 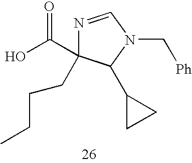 | 90 | 90 |
| 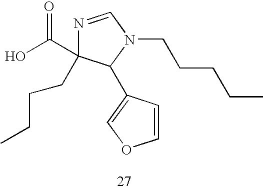 | 92 | 90 |
| 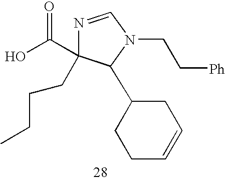 | 74 | 80 |
| 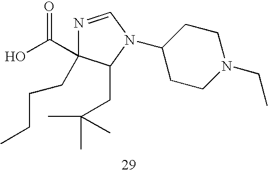 | 54 | 70 |
| 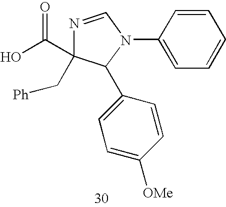 | 67 | 70 |
| 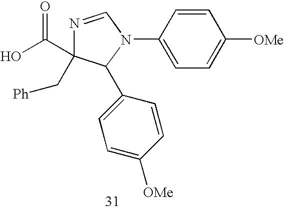 | 73 | 80 |
| 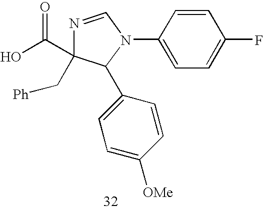 | 60 | 70 |

Following is a typical procedure for the proparation of 2-imidiazolines.

### EXAMPLE 4

### Condensation Reaction of 2-Isocyanohexanoate Wang Resin with p-Anisaldehyde, isobutylamine to Form 2-imidazoline Derivative (23)

To a suspension of 2-isocyano-3-phenyl propinate Wang resin (200 mg, 0.14 mmol) in 2 mL of THF was added solutions of p-anisaldehyde in THF (1.0 M, 0.7 mL), iso-butylamine in MeOH (1.0M, 0.7 mL) and Zinc chloride in ether (1.0 M, 0.25 mL). The resulting slurry was shaken on a orbital shaker at room temperature for 2 days. The resin was then filtered and washed with DMF, DCM, and MeOH several times. The resin was dried at room temperature. Sample of the resin was treated with 25% trifluoroacetic acid in DCM at room temperature for 30 minutes. The resin was filtered. The filtrate was concentrated under vacuum. The resulting residue was analyzed by LC-MS. The analytical data was consistent with expected product. [>90% purity; retention time, 2.51 min; MS (ES) m/z (relative intensity): 333 (M+H⁺, 100)].

We also found that if the aldehyde was replaced with a cyclic ketone, such as cyclohexanone, in the 3-component condensation reaction, an interesting spiro-substituted 2-imidazoline was formed as shown in Scheme 6.

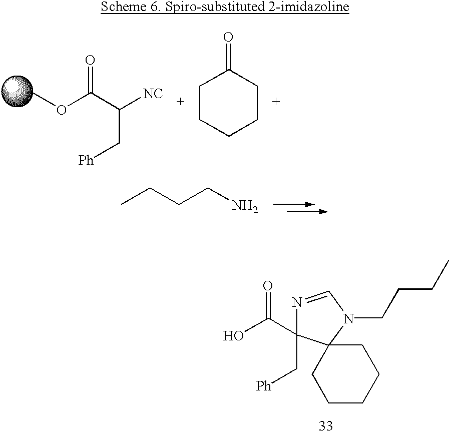

### EXAMPLE 5

### Condensation Reaction of 2-Isocyano-3-phenyl propinate Wang Resin with cyclohexanone, n-butylamine to Form 2-imidazoline Derivative (33)

To a suspension of 2-isocyano-3-phenyl propinate Wang resin (200 mg, 0.14 mmol) in 2 mL of THF was added solutions of cyclohexanone in THF (1.0 M, 0.7 mL), n-butylamine in MeOH (1.0M, 0.7 mL) and Zinc chloride in ether (1.0 M, 0.25 mL). The resulting slurry was shaken on an orbital shaker at room temperature for 2 days. The resin was then filtered and washed with DMF, DCM, and MeOH several times. The resin was dried at room temperature. Sample of the resin was treated with 25% trifluoroacetic acid in DCM at room temperature for 30 minutes. The resin was filtered. The filtrate was concentrated under vacuum. The resulting residue was analyzed by LC-MS. The analytical data was consistent with expected product. [>95% purity; retention time, 3.18 min; MS (ES) m/z (relative intensity): 329 (M+H⁺, 100)].

Synthesis of imidazo[1,2-a]pyridines

Solution phase 3-component condensation reactions involving isocyanides, aldehydes and amines were reported in the literature to afford imidazo[1,2-a]pyridine derivatives. To the best of our knowledge, there was no precedent in the literature, prior to the filing of our provisional patent, that used polymer-bound α-isocyanocarboxylate in this type of reactions. We found that in the presence of Yetterbium trifluoromethanesulfonate, polymer-bound α-isocyanocarboxylate underwent condensation reaction with aldehyde and amine to afford 2-amino imidazo[1,2-a]pyridine derivatives as shown in Scheme 7.

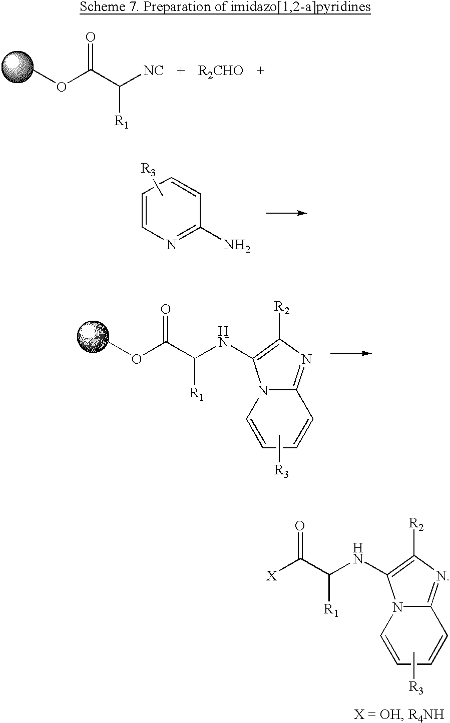

This methodology was successfully utilized for the production of several imidazo[1,2-a]pyridine libraries. Some representative members selected from these libraries are shown in Table 2.

TABLE 2
Imidazo[1,2-a]pyridines From 3-component condensation reactions

| Imidazo[1,2-a]pyridine | Purity(%) |
|---|---|
| 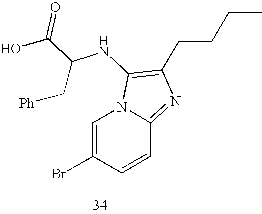 | 80 |
| 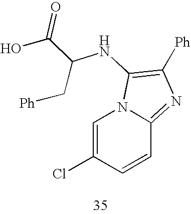 | 80 |
| 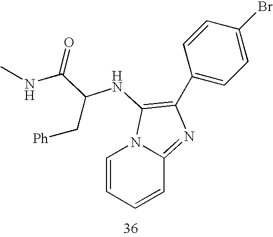 | 80 |
| 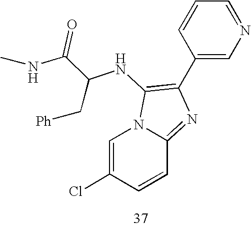 | 80 |
| 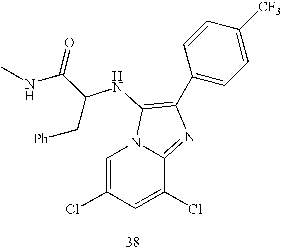 | 80 |
| 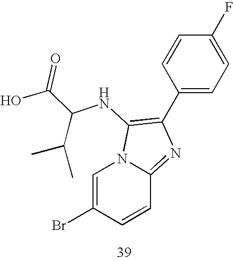 | 80 |
| 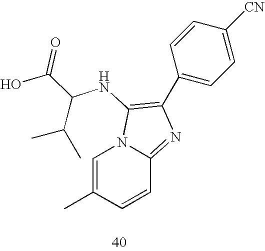 | 90 |
| 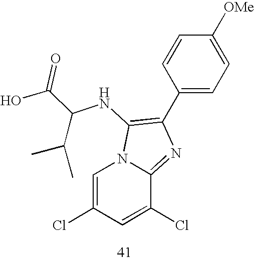 | 90 |
| 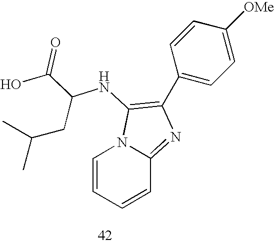 | 90 |
| 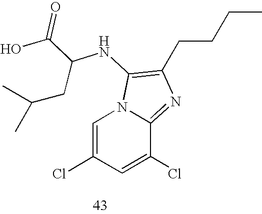 | 90 |
| 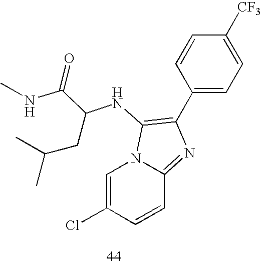 | 90 |
| 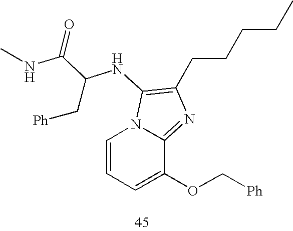 | 80 |

### EXAMPLE 6

### Condensation Reaction of 2-Isocyano-3-phenylbutyrate Wang Resin with 4-fluorobenzaldehyde, 2-amino-5-bromopyridine to Form imidazo[1,2-a]pyridine Derivative (39)

2-Isocyano-3-methylbutyrate Wang resin (200 mg, 0.14 mmol) was mixed with solutions of 4-fluorobenzaldehyde in THF (1.0 M, 0.7 mL), 2-amino-5-bromopyridine in MeOH (1.0M, 0.7 mL) and Yetterbium trifluoromethanesulfonate in MeOH (10%, 0.1 mL) at room temperature for 2 days. The resin was then filtered and washed with DMF, DCM, and MeOH several times. The resin was dried at room temperature. Sample of the resin was treated with 25% trifluoroacetic acid in DCM at room temperature for 30 minutes. The resin was filtered. The filtrate was concentrated under vacuum. The resulting residue was analyzed by LC-MS. The analytical data was consistent with expected product. [>95% purity; retention time, 3.35 min; MS (ES) m/z (relative intensity): 408 (M+H⁺, 100)].

### EXAMPLE 7

### Condensation Reaction of 2-Isocyano-3-phenyl propionate Merrifield Resin with hexanal, 2-amino-3-benzyloxypyridine to Form imidazo[1,2-a]pyridine Derivative (45)

2-Isocyano-3-phenyl propionate Merrifield resin (200 mg, 0.14 mmol) was mixed with solutions of hexanal in THF (1.0 M, 0.7 mL), 2-amino-3-benzyloxypyridine in MeOH (1.0M, 0.7 mL) and Yetterbium trifluoromethanesulfonate in MeOH (10%, 0.1 mL) at room temperature for 2 days. The resin was then filtered and washed with DMF, DCM, and MeOH several times. The resin was dried at room temperature. Sample of the resin was treated with a mixture of THF and 40% aqueous methylamine (4.0 mL) at room temperature overnight. The resin was filtered. The filtrate was concentrated under vacuum. The resulting residue was analyzed by LC-MS. The analytical data was consistent with expected product. [>80% purity; retention time, 3.46 min; MS (ES) m/z (relative intensity): 471 (M+H⁺, 100)].

3. Synthesis of imidazo[1,2-a]pyridine-diazepines

Given the satisfactory results from condensation reactions of polymer-bound α-isocyanocarboxylates, aldehydes, and 2-aminopyridines, we envisioned that, if we use an aldehyde which contains a masked α-amino group, upon deprotection the amino group should be able to cyclize and cleave the ester linkage to afford a tricyclic imidazo[1,2-A]pyridine-diazepine. Indeed, as shown in Scheme 9, when we reacted 2-Isocyano-3-methyl butyrate Wang resin with N-Boc-phenylalaninal, 2-aminopyridine in the presence of Yetterbium trifluoromethanesulfonate, the expected imidazo[1,2-A]pyridine-diazepine derivative (46) was obtained upon TFA/DCM mediated de-protection/cyclization.

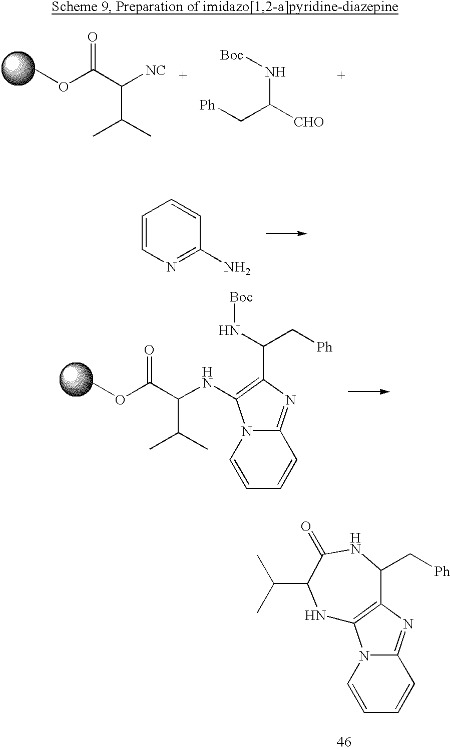

### EXAMPLE 9

### Condensation Reaction of 2-Isocyano-3-methyl butyrate Wang Resin with N-Boc-phenylalaninal, 2-aminopyridine to Form imidazo[1,2-a]pyridine-diazepine Derivative (46)

2-Isocyano-3-methyl butyrate Wang resin (200 mg, 0.14 mmol) was mixed with solutions of N-Boc-phenylalaninal in THF (1.0 M, 0.7 mL), 2-amino-pyridine in MeOH (1.0M, 0.7 mL) and Yetterbium trifluoromethanesulfonate in MeOH (10%, 0.1 mL) at room temperature for 2 days. The resin was then filtered and washed with DMF, DCM, and MeOH several times. The resin was dried at room temperature. Sample of the resin was treated with 25% trifluoroacetic acid in DCM at room temperature for 30 minutes. The resin was filtered. The filtrate was concentrated under vacuum. The resulting residue was analyzed by LC-MS. The analytical data was consistent with expected product. [>95% purity; retention time, 3.81 min; MS (ES) m/z (relative intensity): 335 (M+H⁺, 100)].

4. Synthesis of imidazo[2,1-b]thiazoles

The reaction conditions described for making imidazo[1,2-a]pyridines can also applied for multi-component condensation reactions of polymer-bound α-isocyanocarboxylate, aldehyde and 2-aminothiazole to give imidazo[2,1-b]thiazole as shown in Scheme 8.

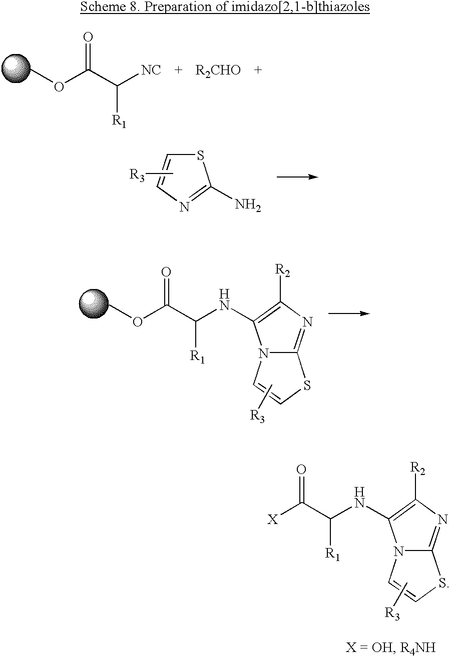

The reactions were readily adopted for library production. Some representative compounds selected from various libraries were shown in Table 3.

TABLE 3
Imidazo[2,1-b]thiazoles From 3-component condensation reactions

| Imidazo[2,1-b]thiazole | Purity(%) |
|---|---|
| 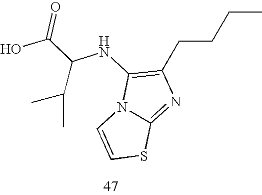 | 95 |
| 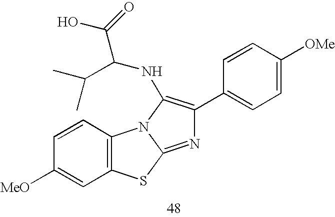 | 80 |
| 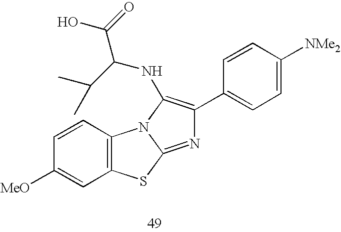 | 80 |
| 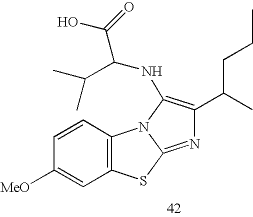 | 80 |
| 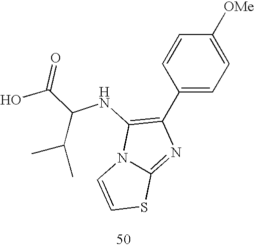 | 75 |
| 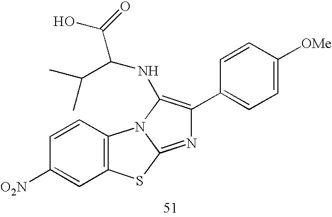 | 80 |

### EXAMPLE 8

### Condensation Reaction of 2-Isocyano-3-methyl butyrate Wang Resin with valeraldehyde, 2-aminothioazole to form imidazo [2,1-b]thiazole Derivative (47)

2-Isocyano-3-methyl butyrate Wang resin (200 mg, 0.14 mmol) was mixed with solutions of valeraldehyde in THF (1.0 M, 0.7 mL), 2-amino-thiazole in MeOH (1.0M, 0.7 mL) and Yetterbium trifluoromethanesulfonate in MeOH (10%, 0.1 mL) at room temperature for 2 days. The resin was then filtered and washed with DMF, DCM, and MeOH several times. The resin was dried at room temperature. Sample of the resin was treated with 25% trifluoroacetic acid in DCM at room temperature for 30 minutes. The resin was filtered. The filtrate was concentrated under vacuum. The resulting residue was analyzed by LC-MS. The analytical data was consistent with expected product. [>95% purity; retention time, 3.45 min; MS (ES) m/z (relative intensity): 296 (M+H⁺, 100)].

As will be understood by those skilled in the art, various arrangements which lie within the spirit and scope of the invention other than those described in detail in the specification will occur to those persons skilled in the art. It is therefor to be understood that the invention is to be limited only by the claims appended hereto.

What is claimed is:

1. A solid phase reaction component for the production of heterocyclic scaffolds in a reaction media, said solid phase component comprising an alpha-isocyanocarboxylate core compound linked to a solid substrate, said solid phase reaction component being insoluble in said reaction media.

2. The solid phase reaction component of claim 1 wherein said core compound has the formula: 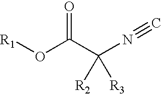
wherein:
R₁ is selected from the group consisting of hydrogen, substituted alkylsilyl, substituted alkyl and substituted alkenyl;
R₂ and R₃ are selected from the group consisting of hydrogen, substituted alkyl, substituted alkenyl, substituted alkenylaryl, substituted alkynyl, substituted aryl, substituted alkylaryl, substituted cycloalkyl substituted alkyl cycloalkyl; or
R₁ and R₂, R₁ and R₃ or R₂ and R₃ taken together are selected from the group consisting of substituted cycloalkyl and substituted saturated heterocycles.

3. The solid phase reaction component of claim 1 wherein said solid substrate is selected from the group support substrates consisting of cellulose, pre-glass beads, silica gels, polypropylene, polyacrylamide, polystyrene, non-cross-linked polystyrene and polyethylene glycol.

4. The solid phase reaction component of claim 3 wherein said solid substrate is polystyrene lightly cross-linked with 1-2% divinylbenzene and optionally grafted with polyethylene glycol and optionally functionalized with amino, hydroxy, carboxy or halo groups.

5. The solid phase reaction component of claim 1 having the formula: 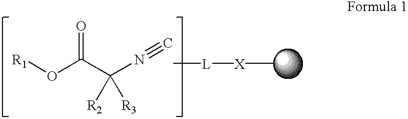
wherein:
the shaded bead is said solid substrate;
L is a linker,
X is an atom or a functional group connecting said solid substrate and said linker L,
R₁ is selected from a group consisting of a covalent bond attached to the L, hydrogen, substituted alkylsilyl, substituted alkyl, substituted alkenyl;
R₂ and R₃ are independently selected from a group consisting of a covalent bond attached to the L, hydrogen, substituted alkyl, substituted alkenyl, substituted alkenylaryl, substituted alkynyl, substituted aryl, substituted alkylaryl, substituted arylalkyl, substituted heteroaryl, substituted heteroarylalkyl, substituted saturated heterocyclyl, substituted cycloalkyl and substituted alkylcycloalkyl; or
R₁ and R₂, or R₁ and R₃, or R₂ and R₃ taken in combination, are substituted cycloalkyl and substituted saturated heterocyclyl;

6. The solid phase reaction component of claim 5 wherein L is a multifunctional chemical monomer in which one functional group reacts with said solid substrate to form a covalent bond with said solid substrate and one other functional group reacts with one of said R₁, R₂, or R₃ of said alpha-isocyanocarboxylate core compound.

7. The solid phase reaction component of claim 6 wherein L is selected from the group consisting of oxygen, ester, amide, sulfur, silicon and carbon.

8. The solid phase reaction component of claim 5 selected from the group consisting of: 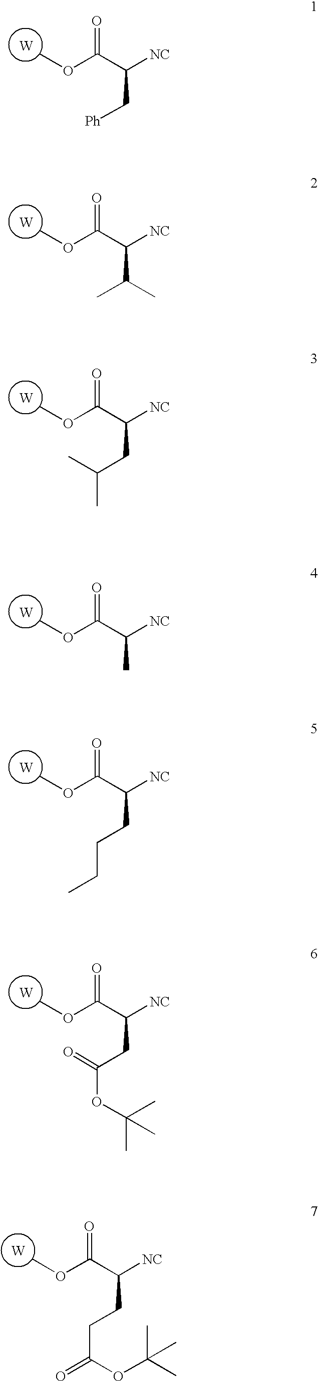 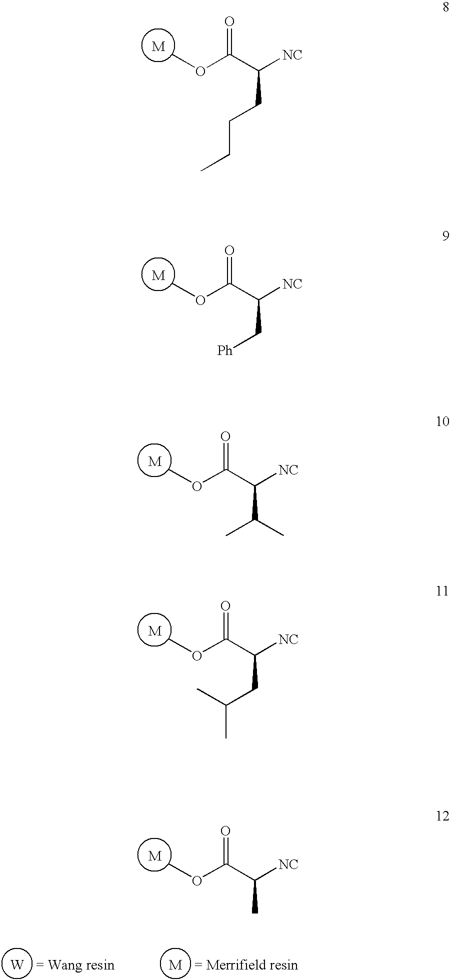
\mathring{W}=Wang resin \mathring{M}=Merrifield resin
where W is substrate consisting of Wang resin and M is a substrate consisting of Merrifield resin.

9. The solid phase reaction component of claim 1 suitable for the synthesis by solid phase reaction of heterocyclic scaffolds, said solid phase reaction component having the formula: 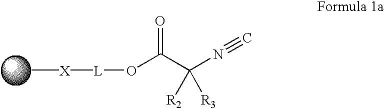
where X, L, R₂ and R₃ are as setout in claim 1.

10. The solid phase reaction component of claim 1 suitable for the synthesis by solid phase reaction of heterocyclic scaffolds, said solid phase reaction component having the formula: 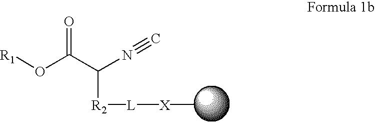
where X, L, R₂ and R₃ are as setout in claim 1.

11. A method for the solid phase synthesis of heterocyclic scaffolds in a reaction media comprising the steps of:
a. forming a solid phase reaction template comprising an alpha-isocyanocarboxylate core compound linked to a solid substrate, said solid phase reaction component being insoluble in said reaction media, said reaction compound having the formula: 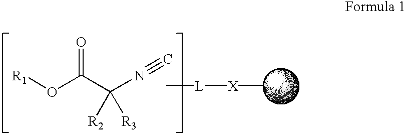
wherein the shaded bead is said solid substrate, L is a linker, X is an atom or a functional group connecting said solid substrate and said linker L, R₁ is selected from the group consisting of hydrogen, substituted alkylsilyl, substituted alkyl and substituted alkenyl,;R₂ and R₃ are selected from the group consisting of hydrogen, substituted alkyl, substituted alkenyl, substituted alkenylaryl, substituted alkynyl, substituted aryl, substituted alkylaryl, substituted cycloalkyl substituted alkyl cycloalkyl or R₁ and R₂, R₁ and R₃ or R₂ and R₃ taken together are selected from the group consisting of substituted cycloalkyl and substituted saturated heterocycles;
b. reacting said solid phase support template in said reaction media to form a reaction product comprising said desired heterocycle scaffold linked to said solid substrate
c. cleaving said reaction product from said solid substrate; and
d. separating said reaction product from said solid support and recovering said reaction product.

12. The method of claim 11 wherein said heterocyclic scaffold is a 2-imidazoline that proceeds according to the following scheme:

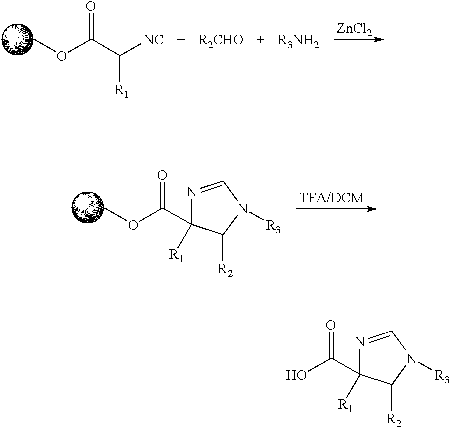

13. The method of claim 11 wherein said heterocyclic scaffold is a 2-imidazoline that proceeds according to the following condensation reaction: 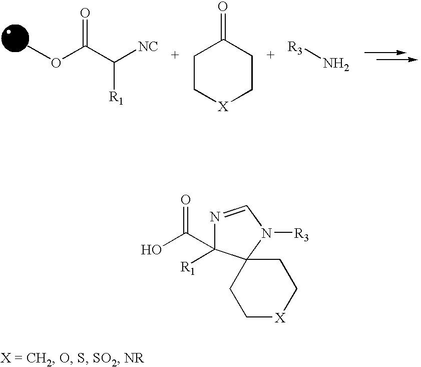
X=CH₂, O, S, SO₂, NR

14. The method of claim 11 wherein said heterocyclic scaffold is an imidazol pyridine that proceeds according to the following reaction:

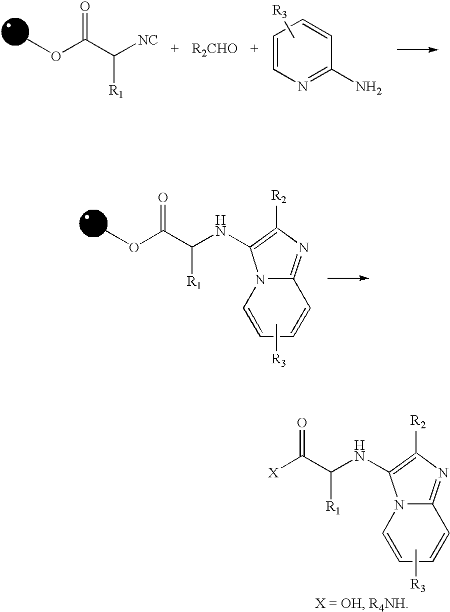

15. The method of claim 11 wherein said heterocyclic scaffold is a imidazol pyridine-diazapine that proceeds according to the following formula:

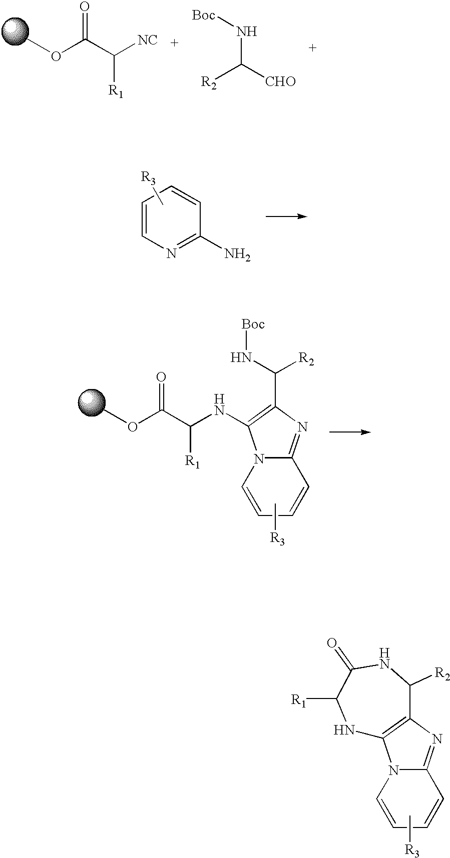

16. The method of claim 11 wherein said heterocyclic scaffold is an imidazol thiazol that proceeds according to the following formula: [chemical structure]
